(19) United States
(12) Patent Application Publication
(10) Pub. No.: US 2007/0298453 A1
(43) Pub. Date: Dec. 27, 2007

(54) Stem Cells

(75) Inventors: Alison Murdoch, Newcastle Upon Tyne (GB); Miodrag Stojkovic, Valencia (ES); Majlinda Lako, Newcastle New Tyne (GB); Yhomas Strachan, Newcastle Upon Tyne (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: University of Newcastle Upon Tyne

(21) Appl. No.: 10/589,229

(22) PCT Filed: Feb. 14, 2005
(86) PCT No.: PCT/GB05/00518

§ 371 Date: Nov. 9, 2006

(30) Foreign Application Priority Data
Feb. 12, 2004 (GB) 0403074.8
May 15, 2004 (GB) 0410910.4
Jan. 15, 2005 (GB) 0500869.3

(51) Int. Cl. C12Q 1/18; C12N 5/08

(52) U.S. Cl. 435/32; 435/366

(57) ABSTRACT

A novel human embryonic stem cell line (hES-NCL1) is described together with a method for culturing a blastocyst and obtaining an embryonic stem cell line therefrom. Also described is the spontaneous partial differentiation of the embryonic stem cell line so obtained to produce fibroblast-like cells which act as an autogeneic feeder system to the stem cells. A novel fibroblast-like cell line hESC-NCL is described.

The present invention relates to the culture of primate embryonic stem cells, to the provision of feeder cells of human origin to support embryonic stem cell culture, and to the provision of fibroblast cells for therapeutic use.

Embryonic stem cells are undifferentiated cells able to proliferate for long periods and which can be induced to differentiate into any type of adult cell.

Human embryonic stem (hES) cells represent a great potential source of various cell types for therapeutic uses, pharmokinetic screening and functional genomics applications (Odorico et al., 2001, Stem Cells 19:193-204; Schuldiner et al., 2001, Brain Res 913:201-205; Zhang et al., 2002, Nat Biotechnol 19:1129-1133; He et al., 2003, Circ Res 93:32-39).

Typically embryonic stem cells are obtained from an embryo at the blastocyst stage (5 to 7 days), by extraction of the inner cell mass (ICM). The ICM is a group of approximately 30 cells located at one end of the internal cavity of the blastocyst. Pluripotent hES cell lines have been obtained from the ICM of Day 5 to 7 blastocysts (Thomson et al., 1998, Science 282:1145-1147; Reubinoff et al., 2000 Nature Biotechnol 18:399-404; Richards et al., 2002, Nature Biotechnol 20:933-936; Hovatta et al., 2003, Hum Reprod 18:1404-1409; Mitalipova et al., 2003, Stem Cells 21:521-526) but to date there have been no reports of obtaining hES cells from older blastocysts due to the difficulty of maintaining the viability of the blastocysts in vitro.

Continuous culture of embryonic stem cells in an undifferentiated (pluripotent) state requires the presence of feeder layers such as mouse embryonic fibroblast (MEF) cells (Thomson et al., 1998, Science 282:1145-1147; Reubinoff et al., 2000, Nat Biotechnol 18:399-404), STO cells (Park et al., 2003, Bio Reprod 69:2007-2017), human foreskin fibroblasts (Hovatta et al., 2003, Hum Reprod 18:1404-14069) human adult fallopian tubal epithelial cells, human fetal muscle and human fetal skin cells (Richards et al. 2002, Nature Biotechnol 20:933-935), or adult skin fibroblast cell lines (Richards et al. 2003, Stem Cells 21:546-556). Alternatively, the culture media can be conditioned by growing the feeder cells in the medium and then harvesting the medium for subsequent stem cell culture (see WO-A-99/20741). Whilst this method is referred to as “feeder-free” culture, nonetheless there is still a reliance on the feeder cells to culture isolated ICMs and to condition the media and hence there is potential for pathogen transmission.

Unfortunately the use of feeder cells for the culture of hES cells limits their medical application for several reasons: xenogeneic and allogeneic feeder cells bear the risk of transmitting pathogens and other unidentified risk factors (Richards et al., 2002, Nat Biotechnol 20:933-936; Hovatta et al., 2003, Hum Reprod 18:1404-1409). Also, not all human feeder cells and cell-free matrices support the culture of hES cells equally well (Richards et al., 2002, Nat Biotechnol 20:933-936; Richards et al., 2003, Stem Cells 21:546-556), and the availability of human cells from aborted foetuses or Fallopian tubes is relatively low. Additionally there are ethical concerns regarding the derivation of feeder cells from aborted human foetuses.

For example, WO-A-03/78611 describes a method of culturing human fibroblasts delivered from aborted human foetuses, typically of 4 to 6 week gestation. The fibroblasts are cultured from the rib region of the embryo and are described as being suitable to support human embryonic stem cell culture. However this method relies upon the donation of aborted foetuses to maintain a supply of fibroblasts. US-A-2002/0072117 and U.S. Pat. No. 6,642,048 describe the production of a human embryonic stem cell line by culturing the ICM of blastocysts and subsequently inducing the embryonic stem cells to form embryoid bodies and to differentiate into mixed differentiated cell populations. Cells having a morphology typical of fibroblasts were selected for use as feeder layers or to condition cell culture media for feeder-free culture. However no markers typical of fibroblasts were noted as being present on these cells.

There remains a need to culture primate embryonic stem (pES) cells, especially hES cells intended for therapeutic use, using only feeder cells of the same species or media conditioned by such feeder cells, to reduce the risk of cross-species pathogen transmission. Additionally, as mentioned above, the use of aborted foetuses as a source of human feeder cells is recognised to be of ethical concern and an alternative source of suitable feeder cells is required.

The present invention provides a novel human embryonic stem (hES) cell line. The novel cell line is termed hES-NCL1. A sample of the hES-NCL1 cell line was deposited in accordance with the Budapest Treaty on 13 Jan. 2005 at the National Institute for Biological Standards and Control (NIBSC), Blanche Lane, South Mimms, Potters Bar Herts., EN6 3QC. The Accession Number allocated to the deposit was P-05-001.

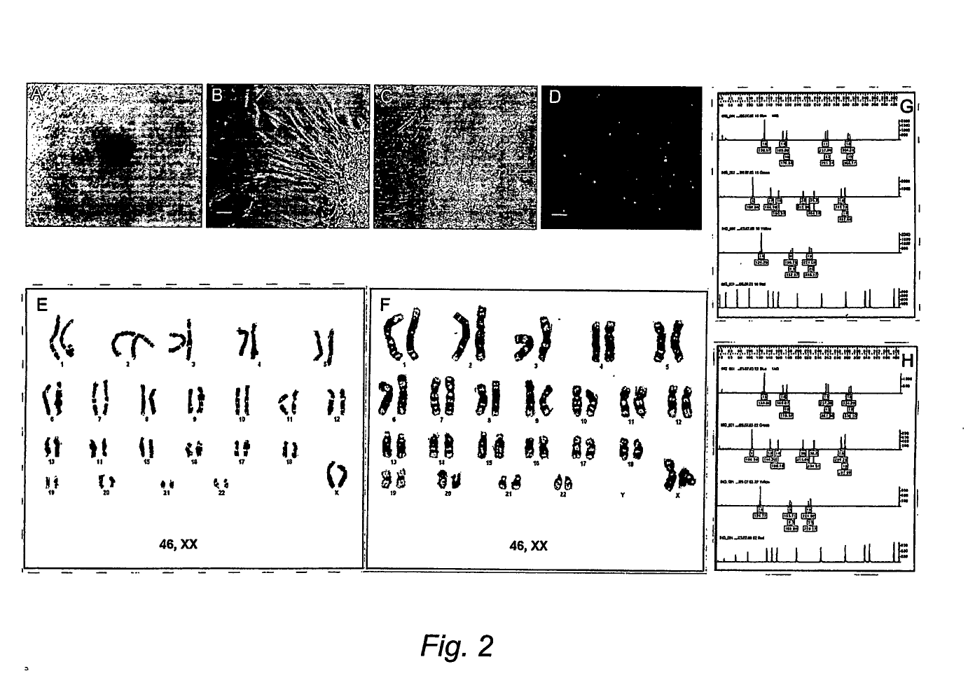

The hES cell line described above was isolated using novel methodology, which forms a further aspect of this invention, and was noted to spontaneously differentiate into fibroblast-like cells in the absence of any trigger and without the formation of embryoid bodies. The fibroblast-like cells so formed expressed the specific fibroblast marker AFSP (anti-fibroblast cell surface specific protein, from Sigma). A photomicrograph of the stained fibroblast-like cells is shown at FIGS. 2B, C, D. The stem cell derived fibroblast-like cells, their formation and their use in culture (as feeder cells or to condition the culture media) of animal embryos (including non-human embryos such as non-human primate embryos as well as human embryos) or embryonic or non-embryonic stem cells (which embryonic or non-embryonic stem cells may be of human or non-human origin), and in therapy forms a further aspect of the present invention and is discussed further below.

In one aspect, the present invention provides a method of culturing a blastocyst, said method comprising exposing said blastocyst to Buffalo rat liver cells or media conditioned thereby for at least 12 hours.

The Buffalo rat liver cells may conveniently be present in the cell culture media or, more preferably, will be used to condition that media.

The blastocyst may be exposed to the Buffalo rat liver cells or media conditioned thereby for a minimum period of 24 hours, 36 hours, 48 hours, 60 hours or 72 hours. We have found that an exposure period of approximately 2 days is sufficient. Where the blastocyst is to be used to generate pluripotent embryonic stem cells, it is desirably exposed to the Buffalo rat liver cells or media conditioned thereby in the period immediately prior to the extraction of cells of the ICM. Benefits may also be obtained from exposing the blastocyst to Buffalo rat liver cells or media conditioned thereby where the blastocyst is intended for implantation as part of IVF treatment.

In more detail, one protocol for culturing a blastocyst according to the present invention comprises:
i) culturing said blastocyst from fertilisation in G1 media;
ii) transferring said blastocyst of step i) to G2.3 media and maintaining said blastocyst in the G2.3 media; and
iii) transferring said blastocyst of step ii) to cell culture media conditioned by Buffalo rat liver cells.

The G1 and G2.3 media referred to above can be obtained from Vitrolife Sweden AB, Kungsbacka, Sweden.

G-1™ is a media designed to support the development of embryos to the 8-cell stage, ie. from pro-cleavage to day 2 or 3. The media contains carbohydrates, amino acids and chelators, as well as Hyaluronan and is bicarbonate buffered. In more detail, the G-1™ media contains:

Alanine
Alanyl-glutamine
Asparagine
Aspartate
Calcium chloride
EDTA
Glucose
Glutamate
Glycine
Hyaluronan
Magnesium sulphate
Penicillin G
Potassium chloride
Proline
Serine
Sodium bicarbonate
Sodium chloride
Sodium dihydrogen phosphate
Sodium lactate
Sodium pyruvate
Taurine
Water for injection (WFI)

G-2™ is a cell culture media to support the development of embryos from around the 8-cell stage to the blastocyst stage. The media contains carbohydrates, amino acids and vitamins, as well as Hyaluronan, and is bicarbonate buffered. In more detail the G-2™ version 3 (ie. G2.3) media contains:

Alanine
Alanyl-glutamine
Arginine
Asparagine
Aspartate
Calcium chloride
Calcium pantothenate
Cystine
Glucose
Glutamate
Glycine
Histidine
Hyaluronan
Isoleucine
Leucine
Lysine
Magnesium sulphate
Methionine
Penicillin G
Phenylalanine
Potassium chloride
Proline
Pyridoxine
Riboflavin
Serine
Sodium bicarbonate
Sodium chloride
Sodium dihydrogen phosphate
Sodium lactate
Sodium pyruvate
Thiamine
Threonine
Tryptophan
Tyrosine
Valine
Water for injection (WFI)

The duration of step i) above may typically be from Day 0 (at fertilisation) to Day 3.

The duration of step ii) above may typically be for 2 or 3 days, that is from Day 3 to Day 5 or 6.

The duration of step iii) above is for a minimum period of 24 hours as described above, but may typically be for 1 to 3 days.

In step iii) a preferred cell culture media consists of Dulbecco's modified Eagle's medium (DMEM, Invitrogen, Paisley, Scotland), optionally supplemented with 15% (v/v) Glasgow medium, and conditioned by Buffalo rat liver cells (see Stojkovic et al., 1995, Biol Reprod 53:1500-1507). Typically conditioning by the Buffalo rat liver cells comprises culturing approximately 75000 Buffalo rat liver cells/cm² in Glasgow medium for 24-36 hours. The media is then recovered and frozen at −20° C. until required.

Using a blastocyst cultured as described above, the ICM can be extracted using routine techniques as late as Day 8, typically by immunosurgery (see Reubinoff et al., 2001, Hum Reprod 10:2187-2194). Blastocysts are cultured for 30 minutes in whole human antiserum (Sigma) diluted 1:5 in DMEM+FCS medium (i.e. 80% Dulbeco's modified Eagle's medium with 10-20% (v/v) fetal calf serum). Furthermore, the blastocysts are washed three times and cultured for another period of approximately 20 minutes in guinea pig complement (1:5). The isolated ICMs can be used for embryonic stem cell culture but could alternatively be implanted into a receptive female as part of an IVF treatment.

For human blastocysts, the blastocyst will have been donated, with informed consent, as being superfluous to IVF treatment. For other (ie. non-human) primates, the ovulation cycle can be controlled by intramuscular injection of prostaglandin or a prostaglandin analogue, and the embryos harvested by a non-surgical uterine flush procedure (see Thompson et al., 1994, J Med Primatol 23:333-336) at day 8 following ovulation. If the blastocyst is unhatched, the zona pellucida is removed by brief exposure to pronase. This step is not required for hatched embryos. The blastocyst is exposed to antiserum for 30 minutes. The blastocyst is then washed three times in DMEM, and exposed to a 1:5 dilution of Guinea pig complement (Gibco) for 20 minutes. After two further washes in DMEM, lysed trophectoderm cells are removed from the ICM by pipette and the ICM plated out on a suitable feeder layer. Embryonic stem cell lines are identified from the cultured ICM cells.

As mentioned above, the novel methodology enables the blastocyst to be cultured at a relatively late stage, day 8. At day 8 the number of cells obtainable from the ICM is considerably increased, but surprisingly these cells retain their pluripotent ability.

The present invention therefore provides a method of producing an embryonic stem cell line, said method comprising:
i) culturing a blastocyst as described above; and
ii) extracting cells of the ICM from said blastocyst and culturing the cells to produce an embryonic stem cell line therefrom.

The reference to culturing the cells of the ICM extracted from the blastocyst in step ii) above includes the published protocols available and is not especially dependent upon any particular culture conditions.

The method of producing stem cells according to the present invention provides a generic and efficient method for the production of primate embryonic stem (pES) cell lines. The pES cell lines may be human embryonic stem (hES) cell lines. An exemplary hES cell line produced by this methodology is the cell line hES-NCL deposited as cell line P-05-001. Alternatively the pES cells may be of non-human origin. The stem cell lines so produced are preferably of clinical and/or GMP grade.

In one embodiment the stem cells of the present invention and/or obtained by the method described above are pluripotent stem cells.

In one embodiment the stem cells of the present invention and/or obtained by the method described above are multipotent stem cells.

In one embodiment the stem cells of the present invention and/or obtained by the method described above are unipotent stem cells.

One suitable medium for the isolation of embryonic stem cells consists of 80% Dulbecco's modified Eagle's medium (DMEM; obtainable from Invitrogen or Gibco) with 10-20% (v/v) fetal calf serum (FCS, Hyclone, Logan, Utah). Optionally the medium may also include one or more of 0.1 mM β-mercaptoethanol (Sigma), up to 1% (v/v) non-essential amino acid stock (Gibco), 1% (v/v) antibiotic, such as penicillin-streptomycin (Invitrogen), and/or 4 ng/ml bFGF (Invitrogen). To date details of several specific media suitable for embryonic stem cell culture have been published in the literature—see for example Thomson et al., 1998, Science 282:1145-1147; Xu et al., 2001, Nature Biotechnol 19:971-974; Richards et al., 2002, Nature Biotechnol 20:933-936; and Richards et al., 2003, Stem Cells 21:546-556.

Feeder cells which may be used for stem cell culture include mouse embryonic stem cells (MEF), STO cells, foetal muscle, skin and foreskin cells, adult Fallopian tube epithelial cells (Richards et al., 2002, Nat Biotechnol 20:933-936; Amit et al., 2003, Biol Reprod 68:2150-2156; Hovatta et al., 2003, Hum Reprod 18:1404-1409; Park et al., 2003, Biol Reprod 69, 2007-2014; Richards et al., 2003, Stem Cells 21:546-556), adult bone marrow cells (Cheng et al., 2003, Stem Cells 21:131-142), or on coated dishes with animal based ingredients with the addition of MEF cell conditioned media (Xu et al., 2001, Nature Biotechnol 19:971-974).

The method of culturing a blastocyst and the method of producing embryonic stem cell lines as described above are both suitable for use with blastocysts of primate origin, including blastocysts of human or non-human origin.

The human embryonic stem cells of the present invention are characterised by at least one of the following;
i) presence of the cell surface markers TRA-1-60, GTCM2, and SSEA-4;
ii) expression of Oct-4;
iii) expression of NANOG;
iv) expression of REX-1; and/or
v) expression of TERT.

In one embodiment at least 2 or more of the characteristics listed above are present, preferably 3 or more of the characteristics are present, especially 4 or more, more preferably all of the above characteristics are present in the stem cells.

The antigen SSEA-4 is a glycolipid cell marker. Specific antibodies to identify this marker are available from the Development Studies Hybridoma Bank, DSHB, Iowa City, Iowa.

The cell surface marker TRA-1-60 is recognised by antibodies produced by hybridomas developed by Peter Andrews of the University of Sheffield (see Andrews et al., “Cell lines from human germ cell tumours” pages 207-246 in Teratocarcinomas and Embryonic Stem Cells: A Practical Approach, Ed. Robertson, Oxford, 1987). TRA1-60 is also commercially available (Chemicon). Both GTCM2 and TG343 are described in Cooper et al., 2002, J. Anat. 200(Pt 3):259-65.

The embryonic stem cell line according to the present invention as described above or which is produced according to the method of the present invention as described above (and specifically the stem cell line hES-NCL1) can be used for screening and/or to produce differentiated cells of specific cell types for therapeutic purposes (e.g. for implantation to replace damaged, diseased or missing tissue). The stem cell lines (e.g. hES-NCL1) can be used to screen agents (e.g. chemical compounds or compositions) for toxicity and/or for therapeutic efficacy (i.e. pharmacological activity).

In a further aspect, the present invention provides a method of screening an agent for toxicity and/or for therapeutic efficacy, said method comprising:
a) exposing an embryonic stem cell line according to the present invention (e.g. hES-NCL1) or obtained by the method described above to said agent;
b) monitoring any alteration in viability and/or metabolism of said stem cells; and
c) determining any toxic or therapeutic effect of said agent.

Additionally, the method of producing a stem cell line according to the present invention as described above, and the stem cell lines produced thereby (e.g. hES-NCL1) may be used in the creation of an embryonic stem cell bank for use in screening and/or to produce differentiated cells of specific cell types for therapeutic purposes. The stem cell bank, which forms a further aspect of the present invention, will consist of a multiplicity of genetically distinct stem cell lines. The stem cell lines forming the stem cell bank will usually be of primate embryonic stem cells such as human embryonic stem cells or non-human embryonic stem cells. The embryonic stem cell bank can be used to screen agents (e.g. chemical compounds or compositions) for toxicity and/or for therapeutic efficacy (i.e. pharmacological activity).

Thus, in a yet further aspect, the present invention provides a method of screening an agent for toxicity and/or for therapeutic efficacy, said method comprising:
a) exposing an embryonic stem cell bank comprising a multiplicity of embryonic stem cell lines according to the present invention or obtained by the method described above to said agent;
b) monitoring any alteration in viability and/or metabolism of said stem cells; and
c) determining any toxic or therapeutic effect of said agent.

As briefly mentioned above, it was noted that the embryonic stem cell line established from a blastocyst cultured as described above according to the present invention spontaneously differentiated into fibroblast-like cells without formation of embryoid bodies. Such spontaneous differentiation into a single cell type was totally unexpected. These fibroblast-like cells then acted as a feeder layer for the remaining undifferentiated embryonic stem cells of the culture. The stem cell derived fibroblast-like cells and the embryonic stem cells supported thereby were autogeneic.

The spontaneous differentiation of hES cells in a feeder-free culture into a mixture of cell types, including fibroblast-like cells, has already been described (see Park et al., 2003, Biol Reprod 69:2007-2014) but in that study the differentiation was observed in the centre of the hES cell colonies. This differs to the present invention where differentiation occurs at the periphery of the colony. Moreover in the present invention only fibroblast-like cells were observed and no other cell types were noted to be present.

In one embodiment the present invention provides a method of producing fibroblast-like cells, said method comprising:
i. providing a stem cell line according to the present invention; and
ii. allowing cells of said stem cell line to differentiate into stem cell derived fibroblast-like cells.

In an alternative embodiment the present invention provides a method of producing fibroblast-like cells, said method comprising:
i) culturing a blastocyst as described above;
ii) extracting cells of the ICM from said blastocyst and culturing the cells to produce an embryonic stem cell line therefrom; and
iii) allowing cells of said embryonic stem cell line to differentiate into stem cell derived fibroblast-like cells.

The stem cell derived fibroblast-like cells are produced without requiring a specific stimulant, e.g. growth factor or change in physical growth conditions (e.g. allowing the cells to become crowded).

One suitable method for obtaining differentiation of the stem cells into fibroblast-like cells was simply to transfer the stem cells to cell culture media in the absence of feeder cells or feeder cell conditioning. The stem cells responded by differentiation of a proportion of the stem cells which then acted as feeder cells for the non-differentiated remaining stem cells. Thus obtaining differentiation into fibroblast-like cells was possible using an extremely easy one-step process, avoiding the need for time-consuming procedures and allowing the differentiation to be fully controlled under in vitro conditions.

The stem cell derived fibroblast-like cells are characterised by a morphology typical of the cell type, ie. long flat cells with an elongated, condensed nucleus. The cytoplasmic processes therein resemble those found in fibroblasts of connective tissue.

The fibroblast-like cells of the present invention are positive for the cell surface marker AFSP. In addition, the identity of hES cells-derived fibroblasts was confirmed by karyotyping and DNA analysis of both stem cells and hES cells-derived fibroblasts. This confirmed that hES cells-derived fibroblasts are autogeneic i.e. of the same origin as the stem cells.

The fibroblast-like cells according to the present invention could be easily immortalised using known techniques to provide a long term source of the cells.

The present invention also provides a novel human embryonic stem cell derived fibroblast-like cell line. The novel fibroblast-like cell line, termed hESCdF-NCL, has been deposited at the European Collection of Cell Cultures (ECACC) on 19 Jan. 2004 under Accession No 04010601.

The fibroblast-like cells and media conditioned by the fibroblast-like cells of the present invention are suitable to support the growth of embryos. The fibroblast-like cells and media conditioned by the fibroblast-like cells of the present invention are alternatively suitable to support the growth of stem cells, especially non-human primate embryonic stem cells or human embryonic stem cells. Other types of stem cells needing the use of feeder cells to survive are also included and particular mention may be made of unipotential and pluripotential stem cells such as adult stem cells, haemapoietic stem cells, mesenchymal stem cells, osteogenic stem cells, chondrogenic stem cells, neuronal stem cells, gonadal stem cells, epidermal stem cells and somatic/progenitor stem cells. Where the fibroblast-like cells of the present invention are used to support human stem cells, the fibroblast-like cells are desirably autogeneic thereto but xenogeneic feeder cells may be used following screening to ensure that they are pathogen-free.

In a further aspect, the present invention provides a self-feeder system for the growth of undifferentiated stem cells, said system comprising
i) culturing a blastocyst as described above, extracting cells of the ICM from said blastocyst and culturing the cells to produce an embryonic stem cell line therefrom, or providing a stem cell line according to the present invention; and
ii) allowing some of the cells of said embryonic stem cell line to differentiate into stem cell derived fibroblast-like cells whilst the remainder of the cells of said embryonic stem cell line remain in an undifferentiated pluripotent, multipotent or unipotent state, whereby said stem cell derived fibroblast-like cells act as autogeneic feeder cells for said stem cells.

The fibroblast-like cells may be used directly as feeder cells to support stem cell culture (eg are grown as a confluent surface in contact with the stem cells) or may be used to condition media for use in stem cell culture. Generally, where the media is to be conditioned, the fibroblast-like cells are grown in the media for a predetermined period of typically 24 hours, although periods of up to a maximum of 9 days may be used, before the media is removed and transferred to the stem cells.

There are several advantages for using hES cells derived fibroblasts as feeder cells: i) feeder derived from hES cells offers more secure autogeneic/genotypically homogenous system for prolonged growth of undifferentiated hES cells, ii) feeders differentiated from first clinical-grade hES cell line could be used worldwide as initial monolayer for growth of isolated ICMs to eliminate transfer of pathogens, iii) the long proliferation time of already derived hES cell lines allows screening for viral contamination, iv) medium conditioned by hESdF can be used for feeder-free growth of hES cells thus avoiding potential viral transfer from the MEF conditioned media used to date, v) due to the low bioburden, embryonic tissues perform better support in vitro than adult tissues (see Richards et al., 2003, Stem Cells 21:546-556), vi) derivation and culture of hESdF is fully controlled and not time consuming, vii) derived feeder cells could be easily immortalized to provide a long-term source of this tissue, viii) in vitro studies on cell-to-cell contacts and identification of isolated soluble factors could significantly improve cell-culture, cell-transplantation and tissueengineering avoiding at the same time expensive tissue-biopsy and unnecessary sacrifice of animals.

Accordingly, the present invention further provides a method of culturing a primate embryonic stem cell line, such as a human embryonic stem cell line, to maintain the viability of eggs prior to or during fertilisation and/or to culture blastocysts or embryos intended for implantation into a receptive female to establish a pregnancy (i.e. as part of an IVF procedure). The method comprises providing fibroblast-like cells according to the present invention or obtained by the method described above as feeder cells or to condition the cell culture media. Advantageously the fibroblast-like cells selected will be obtained from an embryonic stem cell line of the same origin or species, and will be previously screened to ensure pathogen-free status. This approach enables the complete elimination of animal ingredients for the culture of undifferentiated hES cells and avoids the potential of viral transfer which may occur when MEF conditioned media or conditioned media from other feeders is used for stem cell culture.

We have found that the use of the fibroblast-like cells obtained according to the present invention (e.g. hESCdF-NCL) as feeder cells or to condition the culture media enables the undifferentiated culture of the embryonic stem cells. It is anticipated that a similar ability will be obtained using other stem cell types. This is highly significant for the long term maintenance of such cell lines and also has the advantage that the extended culture period possible for the undifferentiated stem cell line enables the cell line to be screened for any potential pathogen (e.g. viral contamination).

Alternatively, the fibroblast-like cells can be used for therapy, for example to assist regeneration of wounds requiring fibroblast presence.

The presence of fibroblast cells, without contamination of other cell types is of particular advantage in therapy. One example of the use of the fibroblasts according to the present invention is the generation of skin grafts for use in treating wounds (for example burns) or in cosmetic or regenerative surgery.

The present invention will now be further described with reference to the following examples and figures, in which:

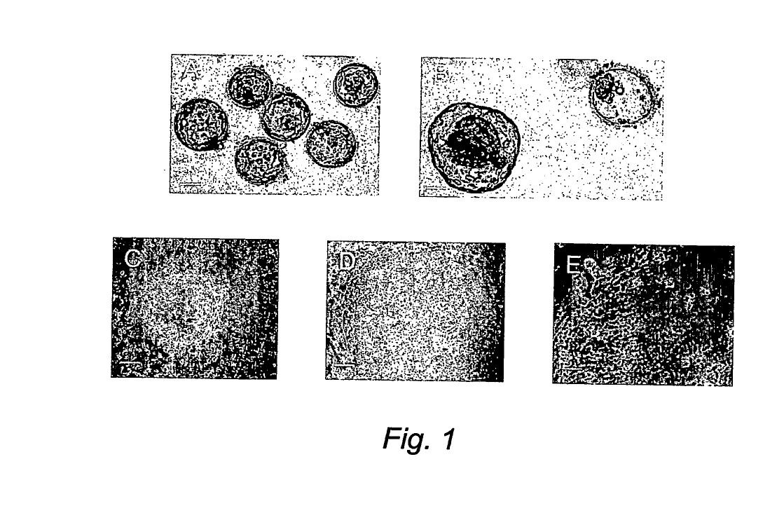

FIG. 1. Morphology of human blastocysts and hES cells. Day 6 blastocysts (A) and hatched Day 8 blastocysts (B). Note the presence of very well organised inner cell mass in Day 8 blastocyst recovered after three-step in vitro culture. Inner cell mass cells (C) grown on irradiated MEF 4 days after immunosurgery. Primary hES cells colony (D) grown on inactivated MEF cells. Same colony at high magnification (E). Bars: 50 Mm (A-D); 100 μm (E).

FIG. 2. Morphology and characterisation of hES cells-derived fibroblasts. Undifferentiated hES cells (A). Peripheric differentiation of hES cells into fibroblast-like cells in feeder-free conditions (B). Phase (C) and fluorescence (D) microscopy of hES cells-derived fibroblasts using AFSP antibody. Normal 46+XX karyotypes of hES cells (E) and hES cells-derived fibroblasts (F). Microsatellite analysis of hES cells (G) and hES cells-derived fibroblasts (H). Bars: 50 μm (A, C, D), 100 μm (B).

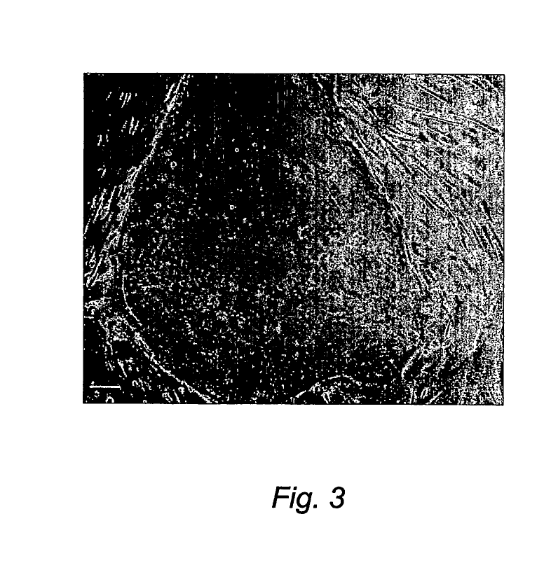

FIG. 3. Morphology of frozen/thawed hES-NCL1 colony cultured on frozen/thawed hES cell-derived fibroblasts. Bar: 50 μm.

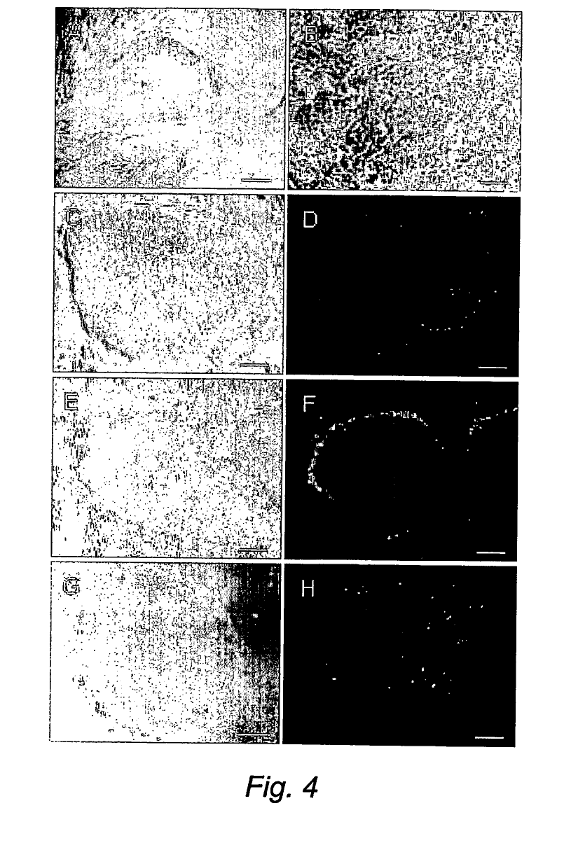

FIG. 4. Morphology and characterisation of hES-NCL1 cells grown on γ-irradiated hESdF monolayer (A-F) or feeder-free (G, H). (A) Five days old vitrified hES-NCL1 colony cultured on frozen/thawed hESdF (passage 8). (B) Higher magnification of the same hES colony. Note typical morphology of hES cells i.e. small cells with prominent nucleoli. HES cells grown on hESdF stained with antibody recognising the TRA1-60 (D) and SSEA-4 (F) epitopes. HES cells grown on Matrigel (G) with addition of hESdF conditioned medium stained with antibody recognising the GTCM2 epitope (H). Bars: 200 μm (A, E-H); 50 μm (B); 100 μm (C, D).

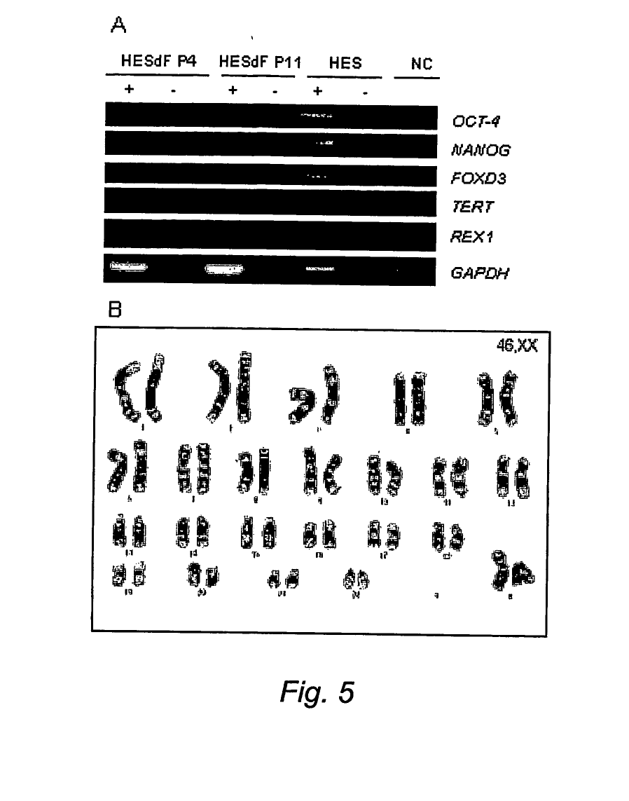

FIG. 5. Characterisation and karyotyping of hES-NCL1 cells grown on hESdF monolayer. RT-PCR analysis of undifferentiated hES cells grown on inactivated hESdF cells (A). PCR products obtained using primers specific for OCT-4, NANOG, FOXD3, TERT, REX1 and GAPDH. HES cells (passage 31) grown on hESdF (passage 11) show normal female karyotype (46, XX) (B).

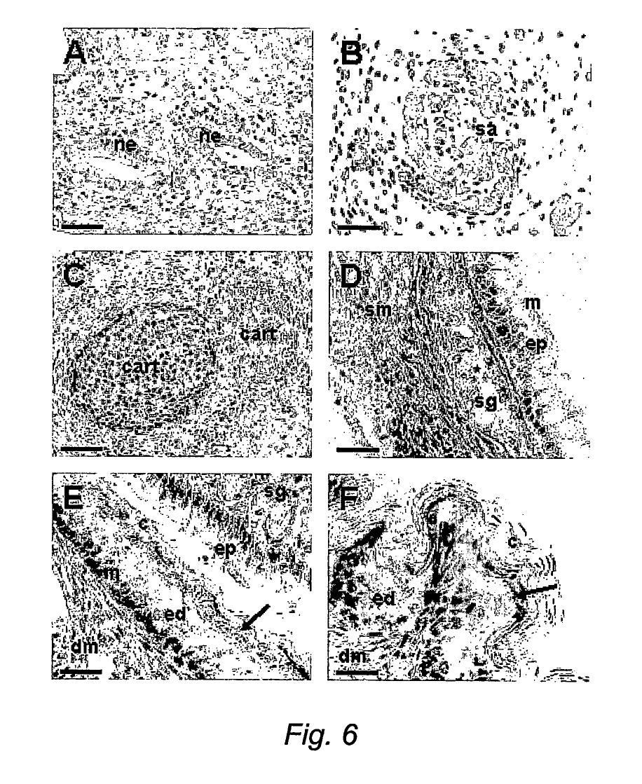

FIG. 6. Histological analysis of teratomas formed from grafted colonies of hES cells grown on inactivated hESdF in testis (A-C) and kidney (D-F) of SCID mice. (A) neural epithelium (ne); (B) aggregation of glandular cells with characteristic appearance of secretory acini (sa); (C) cartilage (cart); (D) wall of respiratory passage showing epithelium (ep), submucosa (sm), submucosal glands (sg). Epithelium contains occasional ciliated cells and numerous goblet cells secreting mucin (m); (E) Two types of epithelia: respiratory (top), keratinised skin (bottom). Submucosal glands (sg) located beneath pseudostratified ciliated (in parts) epithelium (ep). Structures of the skin include epidermis (ed), dermis (dm) and cornified layer (c). Note that the stratum granulosum (arrow) is characterised by intracellular granules which contribute to the process of keratinisation. Occassional mitotic indices (m) are seen in the basal layer; (F) High magnification image of skin, showing greater detail of dermis (dm), epidermis (ed) and cornified layer (c). Again the stratum granulosum is visible (arrow). Scale bars: (A, B, C) 100 μm; (D, E) 25 μm; (F) 17.5 μm.

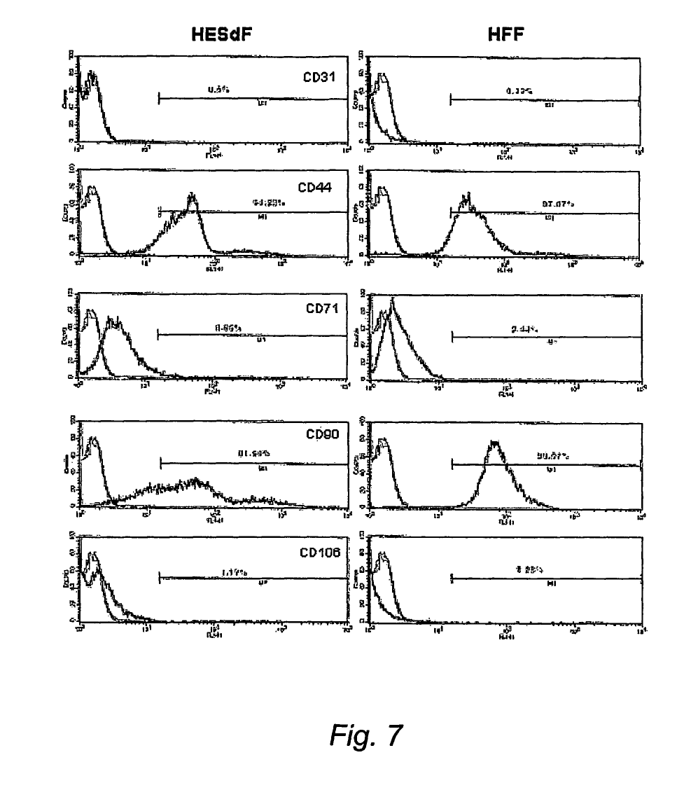

FIG. 7. Flow cytometry analysis of hESdF (left panel) and human foreskin fibroblasts (HFF, right panel) for the presence of CD31, CD44, CD71, CD90 and CD106. The bold (red) line represents the staining with the isotype control and the grey (green) line staining with specific antibodies.

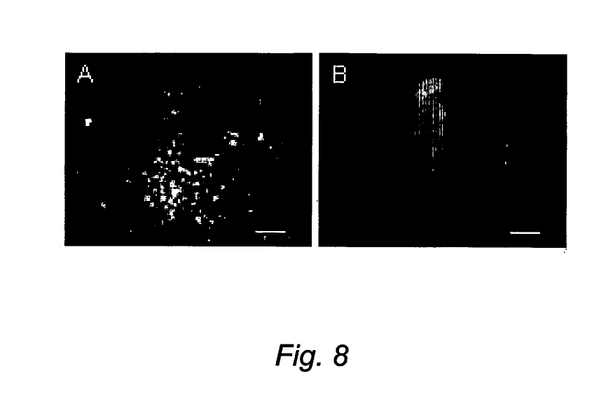

FIG. 8. Spontaneous differentiation of hES-NCL1 cells grown on hESdF and then in feeder-free conditions. hES-NCL1 differentiate into neuronal (A) and smooth muscle (B) cells demonstrating differentiation into cells of ectoderm and mesoderm, respectively. Green: cells stained with nestin antibody (A) and smooth muscle actin antibody (B). Red: cell-nuclei stained with propidium iodide. (A) shows small areas of red and green staining dispersed across the cells in a check-like pattern. (B) shows all cells stained green. Scale bars: 100 μm (A) and 50 μm (B).

### EXAMPLES

### Material and Methods

Culture of embryos. Two day old human embryos, produced by in vitro fertilization (IVF) for clinical purposes, were donated by individuals after informed consent and after Human Fertilisation and Embryology Authority (HFEA, UK) approval. Until Day 3 (IVF=Day 0), 11 embryos were cultured in G1 medium and transferred to G2.3 medium (both G1 & G2.3 from Vitrolife, Kungsbacka, Sweden) until day 6. Day 6 recovered blastocysts were cultured in Dulbecco's modified Eagle's medium (DMEM, Invitrogen, Paisley, Scotland) supplemented with 15% (v/v) Glasgow medium conditioned by Buffalo rat liver cells which has been used successfully for the long-term culture of bovine embryos, termed G-BRLC media (Stojkovic et al., 1995, Biol Reprod 53:1500-1507). On Day 8 ICMs were isolated by immunosurgery as previously described (Reubinoff et al., 2001, Hum Reprod 10:2187-2194).

Cell-number analysis. We investigated whether our three-step embryo culture supported development of Day 8 blastocysts and whether these blastocysts posses more ICM cells than Day 6 blastocysts. Eleven isolated ICMs from Day 6 blastocysts (5 blastocysts and 6 expanded blastocysts) and 13 ICMs from Day 8 blastocysts (7 expanded and 6 hatching or hatched blastocysts) were analysed using 1.5 μg/ml 4′-6-diamidino-2-phenylindole (DAPI, Sigma, St. Louis, Mo.) labelling as previously described (Spanos et al., 2000, Biol Reprod 63:1413-1420).

Derivation of hES cells. Initially, isolated ICMs were cultured on γ-irradiated MEFs monolayer (75.000 cell/cm²) and DMEM supplemented with 10% (v/v) Hyclone defined fetal calf serum (FCS, Hyclone, Logan, Utah) for 10 days. After 17 days, the hES cell colony was mechanically dispersed into several small clumps which were cultured on a fresh MEF layer with ES medium containing Knockout-DMEM (Invitrogen), 100 μM β-mercaptoethanol (Sigma), 1 mM L-glutamine (Invitrogen), 100 mM non-essential amino acids, 10% serum replacement (SR, Invitrogen), 1% penicillin-streptomycin (Invitrogen) and 4 ng/ml bFGF (Invitrogen). ES medium was changed daily. Human embryonic stem cells were passaged by incubation in 1 mg/ml collagenase IV (Invitrogen) for 5-8 minutes at 37° C. or mechanically dissociated and then removed to freshly prepared MEF or hES cells-derived feeders.

Recovery of hES cell-derived fibroblasts. Once a stable stem cell line was established, hES cells were transferred into feeder-free T-25 flasks (Iwaki, Asahi, Japan), using DMEM supplemented with 10% FCS at 37° C. in a 5% CO₂ atmosphere. After one week the stem cell derived fibroblast-like cells were transferred into T-75 flasks (Iwaki) and cultured for a further 3 days to produce a confluent primary monolayer of hES cells-derived fibroblasts.

Immunocytochemical analysis of hES cells and hES cells-derived fibroblasts. Live staining was performed by adding primary antibodies (TRA1-60 and TRA1-81, a kind gift from Prof. P. Andrews (University of Sheffield, UK) (but also available commerically from Chemicon); SSEA-4, SSEA-4 (MC-813-70) from Developmental Studies Hybridoma Bank, DSHB, Iowa City, Iowa; GCTM-2 and TG343, both a kind gift from Dr. M. Pera (Monash Institute of Reproduction and Development, Clayton, Australia); anti-fibroblast surface protein, AFSP from Sigma) to hES cells and hES cells-derived fibroblasts for 20 minutes at 37° C. The primary antibodies were used at the following dilutions: TRA-1-60—1:10; TRA1-81—1:10; SSEA-3—1:4; SSEA-4—1:5 (Henderson et al., 2002, Stem Cells 20:239-337); GCTM-2—1:2; AFSP—1:50 (Ronnov-Jessen, 1992, Histochem Cytochem 40:475-486). TG343 at 1:2 (Cooper et al., 2002, J Anat 200:259-265) was used to label cells grown on MEF feeder cells. The samples were gently washed three times with ES medium before being incubated with the 1:100 secondary antibodies (anti mouse IgG and anti mouse IgM, both Sigma) conjugated to fluorescein isothiocyanate (FITC) at 37° C. for 20 minutes. The samples were again washed three times with ES medium and subjected to fluorescence microscopy. For the Oct4 immunostaining hES cells were fixed in 3.7% formaldehyde BDH, Coventry, UK for 20 minutes at room temperature followed by incubation in 3% hydrogen peroxide for 10 minutes. The hES cells were permeabilised with 0.2% Triton×100 (Sigma) diluted in 4% sheep serum (Sigma) for 30 minutes at 37° C. The ES colonies were incubated with the primary antibodies (Oct4 from Santa Cruz Biotechnologies, Heidelberg, Germany, final concentration 10 μg/ml for 30 minutes at room temperature. The ES colonies were washed twice with PBS for 5 minutes and then incubated with the secondary antibody (rat anti mouse immunoglobulin (DAKO, Cambridgeshire, UK) used at 1:100 dilution) for 30 minutes at room temperature. After that, hES cells were washed again with PBS, incubated with ABC/HRP solution for 25 minutes at room temperature and washed again with PBS. The detection was carried out by incubation with DAB peroxidase (Enzo Life Sciences, NY) solution at room temperature for 1 minute. Final washes were done with distilled water. The bright field and fluorescent images were obtained using a Zeiss microscope and the AxioVision software (Carl Zeiss, Jena, Germany).

Comparison of hES cells-derived fibroblasts with human foreskin fibroblasts. To identify the nature of feeder cells, hESdF were compared with human foreskin fibroblasts (HFF; ATCC, Teddington, UK) using flow-cytometry analysis. Briefly, hESdF were harvested using 0.05% Trypsin/0.53M EDTA (Invitrogen, Paisley, Scotland) and suspended in staining buffer (PBS+5% FCS) at concentration 10⁶ cells/ml. Hundred μl of the cell suspension was stained with 0.2 μg of CD31 (PECAM-1), CD71 (Transferrin receptor), CD90 (Thy-1), and CD106 (VCAM-1) antibodies (all available from BD Biosciences, Oxford, UK) at 4° C. for 20 minutes. Three washes in staining buffer were carried out before staining with secondary antibody, goat anti-mouse Ig-FITC (Sigma, Dorset, UK) used at 1:512 dilution at 4° C. for 20 minutes. Cells were washed again three times and resuspended in staining buffer before being analysed with FACS Calibur (BD) using the CellQuest software. 10,000 events were acquired for each sample and propidium iodide staining (1 μg/ml) was used to distinguish live from dead cells.

Karyotype analysis of hES cells and hES cells-derived fibroblasts. The karyotype of hES cells and hES cells-derived fibroblasts was determined by standard G-banding procedure. A suitable protocol is available at: http://www.s1h.wisc.edu/cytogenetics/Protocols/Staining/G-Banding.html

Reverse Transcription (RT)-PCR analysis. The reverse transcription was carried out using the cells to cDNA II kit (Ambion, Huntingdon, UK) according to manufacturer's instructions. In brief, hES cells were submerged in 100 μl of ice-cold cell lysis buffer and lysed by incubation at 75° C. for 10 minutes. Genomic DNA was degraded by incubation with DNAse I for 15 minutes at 37° C. RNA was reverse transcribed using M-MLV reverse transcriptase and random hexamers following manufacturer's instructions. PCR reactions were carried out using the following primers (Seq ID Nos 1 to 12):

OCT4 (F):

(SEQ ID No. 1)

5′-GAAGGTATTCAGCCAAAC-3′;
OCT4 (R):

(SEQ ID No. 2)

5′-CTTAATCCAAAAACCCTGG-3′;
REX1 (F):

(SEQ ID No. 3)

5′-GCGTACGCAAATTAAAGTCCAGA-3′;
REX1 (R):

(SEQ ID No. 4)

5′-CAGCATCCTAAACAGCTCGCAGAAT-3′;
NANOG (F):

(SEQ ID No. 5)

5′-GATCGGGCCCGCCACCATGAGTGTGGATCCAGCTTG-3′;
NANOG (R):

(SEQ ID No. 6)

5′-GATCGAGCTCCATCTTCACACGTCTTCAGGTTG-3′;
FOXD3F:

(SEQ ID No. 7)

5′-GGAGGGAGGGGGCAATGCAC-3′;
FOXD3R:

(SEQ ID No. 8)

5′-CCCCGAGCTCGCCTACT-3′;
TERT (F):

(SEQ ID No. 9)

5′-CGGAAGAGTGTCTGGAGCAAGT-3′;
TERT (R):

(SEQ ID No. 10)

5′-GAACAGTGCCTTCACCCTCGA-3′;
GAPDH (F):

(SEQ ID No. 11)

5′-GTCAGTGGTGGACCTGACCT-3′;
GAPDH (R):

(SEQ ID No. 12)

5′-CACCACCCTGTTGCTGTAGC-3′.

Note that (F) and (R) refer to the direction of the primers and designate forward and reverse direction respectively.

PCR products were run on 2% agarose gels and stained with ethidium bromide. Results were assessed on the presence or absence of the appropriate size PCR products. Reverse transcriptase negative controls were included to monitor genomic contamination.

DNA Genotyping of hES cells and hES cells-derived fibroblasts. Total genomic DNA was extracted from both hES cells and hES cells-derived fibroblasts. DNA from both samples was amplified with 11 microsatellite markers: D3S1358, vWA, D16S539, D2S1338, Amelogenin, D8S1179, D21S11, D18S51, D19S433, TH01, and FGA (Chen Y et al., 2003, Cell Res. 2003 August; 13(4):251-63. full paper available at http://www.cell-research.com/20034/2003-116/2003-4-05-ShengHZ.htm) and analysed on an ABI 377 sequence detector using Genotype software (Applied Biosystems, Foster City, Calif.).

Growth of hES cells on hESdF. HES-NCL1 cells were grown on γ-irradiated hESdF monolayer (75.000 cells/cm²) in ES medium containing Knockout-DMEM (Invitrogen), 100 μM β-mercaptoethanol (Sigma), 1 mM L-glutamine (Invitrogen), 100 mM non-essential amino acids, 10% serum replacement (SR, Invitrogen), 1% penicillin-streptomycin (Invitrogen) and 4 ng/ml bFGF (Invitrogen). ES medium was changed daily. HES cells were passaged every 4-5 days by incubation in 1 mg/ml collagenase IV (Invitrogen) for 5-8 minutes at 37° C. or mechanically dissociated and then removed to plates with freshly prepared hESdF.

Recovery of hESdF-conditioned medium. Mitotically inactivated HESdF were cultured in T-25 flask with addition of ES medium for 10 days. hESdF-conditioned medium was collected every day and then frozen at −80° C.

Growth of hES cells in feeder-free system using hESdF-conditioned medium. hES cells were passaged and then removed to plates precoated with Matrigel (BD, Bedford, Mass.) (Xu et al., 2001, Nat Biotechnol 19:971-974). ES media conditioned by hESdF was changed every 48 hours.

Cryopreservation of hES cells and hESdF. To see whether frozen-thawed hESdF still support undifferentiated growth of cryopreserved hES cells, hESdF were frozen at −80° C. using FCS supplemented with 10% (v/v) dimethyl sulfoxide (Sigma). Clumps of hES cells were frozen or vitrified using protocol as previously described (see Reubinoff et al., 2001, Hum Reprod 10:2187-2194). Mitotic inactivation by using mitomycin C could alternatively be used.

Tumor formation in severe combined immunodeficient (SCID) mice (Stefan). Ten to fifteen clumps with approximately 3000 hES cells in total were injected in kidney capsule, subcutaneously in flank or in the testis. After 21-90 days, mice were sacrificed, tissues were dissected, fixed in Bouins overnight, processed and sectioned according to standard procedures and counterstained with either haematoxylin and eosin or Weigerts stain. Sections were examined using bright field light microscopy and photographed as appropriate.

All procedures involving mice were carried out in accordance with institution guidelines and institution permission.

Statistical analysis. Cell numbers of Day 6 and Day 8 ICMs were compared using Wilcoxon rank-sum test. The data are presented as mean ±standard deviation.

In vitro differentiation of hES cells. Colonies of hES-NCL1 passage 21 were grown in feeder-free conditions in ES medium. After 5 to 14 days spontaneous differentiation was observed and differentiated cells were passaged and cultured under same conditions. Cells were fixed in 4% paraformaldehyde in PBS (Sigma) for 30 minutes and then permeabilised for additional 10 minutes with 0.1% Triton X (Sigma). The blocking step was 30 minutes with 2% FCS in PBS. Cells were incubated with antibody against nestin (1:200; Chemicon) or human alpha smooth muscle actin (1:50; Abcam, Cambridge, UK) for additional 2 hours. Each antibody was detected using corresponding secondary antibodies conjugated to FITC. The nuclei of cells were stained using propidium iodide for 5 minutes.

Results

Traditionally early blastocysts (Day 6) have been used for the derivation of human ES cell line. We developed a three-step culture system (see Materials and Methods) which supports successfully the development of late (Day 8) blastocysts. Analysis of cell numbers of ICMs revealed that Day 8 blastocysts possess significantly (P<0.01) more ICM cells than Day 6 blastocysts (51.3±9.6 vs. 36.8±11.9, respectively). In view of this result we used day 8 blastocysts to derive human ES cell lines. Of the 11 Day 2 donated embryos, 7 (63.6%) blastocysts developed to Day 6. All 7 of these blastocysts expanded or hatched on Day 8 after transfer to G-BRLC medium. After isolation of ICMs by immunosurgery, 3 primary hES cell colonies showed visible outgrowth and one stable hES cell line (ICL-NCL1) was successfully derived (FIGS. 1C-E).

When the hES cells were cultured in the absence of feeder cells they spontaneously differentiated into fibroblast-like cells, ie. long, flat cells with elongated, condensed nucleus. We confirmed that the differentiated cells were fibroblasts by staining with a specific antibody to fibroblast surface protein (AFSP) (FIGS. 2C and D). Karyotyping of the hES cells and hES cells-derived fibroblasts revealed that both samples are normal female (46+XX, FIGS. 2E and F). Microsatellite analysis revealed that the hES cells and hES cells-derived fibroblasts are indistinguishable from each other and should be considered as autogenic (see FIG. 2G, 2H). We now have several batches of fresh and frozen/thawed serially expanded hES cells-derived fibroblasts which support hES cell culture even after the twelfth passage but they are optimal between second and eighth passages. Flow-cytometry (FIG. 7) revealed that very few cells showed expression of mesenchymal cell specific markers CD106 (V-CAM1) and CD71 (transferring receptor) and none expressed the endothelial specific cell marker CD31 (PECAM-1). On the contrary, 94% and 82% of the hESdF cells were stained with the CD44 and CD90 (THY-1) antibodies, respectively. Both antibodies were also presented in human foreskin fibroblasts (HFF; FIG. 7).

The hES-NCL1 line has been cultured on hES cell derived fibroblasts (hESdF) for over 35 passages and on Matrigel with hESdF conditioned medium for 13 passages. We found that hES cell colonies grown on hES cell derived fibroblasts were dense, compact and suitable for mechanical passaging with typical morphology of hES cells (FIG. 4). Characterisation studies demonstrated that hES cells cultured on hES cells-derived fibroblasts or Matrigel with addition of hESdF-conditioned medium expressed specific surface markers: GTCM2, TRA1-60 and SSEA4, and (FIG. 4A-H) and were positive for the expression of OCT-4, NANOG, FOXD3, REX-1 and TERT by RT-PCR (FIG. 5A). Expression of TG343 was also found in hES cells grown on mouse feeder cells, and whilst not tested in the hESdf grown cells would be expected to be present. The fibroblast-like cells also expressed the telomerase reverse transcriptase (TERT) and REX1 in early passages but none of the other ES cell specific markers. Human ES cells grafted into SCID mice consistently developed into teratomas demonstrating the pluripotency of hES-NCL1 cells grown on hESdF. Teratomas were primarily restricted to the site of injection and their histological examination revealed advanced differentiation of structures representative of all three embryonic germ layers, including cartilage, skin, muscle, primitive neuroectoderm, neural ganglia, secretory epithelia and connective tissues (FIG. 6). When hES-NCL1 cells were cultured in absence of feeders and Matrigel, spontaneous differentiation into neuronal (FIG. 8A) and smooth muscle (FIG. 8B) cells was observed.

1-46. (canceled)

47. The stem cell line hES-NCL1 deposited at NIBSC under Accession No. P-05-001.

48. An embryonic stem cell bank, comprising a plurality of genetically distinct stem cell lines, including the stem cell line of claim 47.

49. A method of screening an agent for toxicity or for therapeutic efficacy, or both, the method comprising:
exposing the stem cell line of claim 47 to the agent being screened;
monitoring any alteration in viability or metabolism, or a combination thereof, of the cells of the stem cell line at the time of, or following, exposure to the agent; and
determining any toxic or therapeutic effect of the agent on the cells of the stem cell line.

50. A method of screening an agent for toxicity or for therapeutic efficacy, or both, the method comprising:
exposing an embryonic stem cell bank of claim 48 to the agent being screened;
monitoring any alteration in viability or metabolism, or a combination thereof, of the cell lines in the embryonic cell bank at the time of, or following, exposure to the agent; and
determining any toxic or therapeutic effect of the agent on the cell lines.

51. A method of producing fibroblast-like cells, the method comprising:
providing the stem cell line of claim 47; and
allowing cells of the stem cell line to differentiate into stem cell-derived fibroblast-like cells.

52. The method of claim 51, wherein the fibroblast-like cells are produced for a therapeutic purpose.

53. A method of culturing cells, comprising culturing the cells in the presence of the fibroblast-like cells obtained by the method of claim 51; or conditioning cell culture media in the presence of the fibroblast-like cells and then culturing the cells therein; or culturing the cells in the conditioned cell culture media and in the presence of a population of the fibroblast-like cells.

54. The method of claim 53, wherein the cells being cultured comprise stem cells.

55. The method of claim 51, comprising using no specific stimulant for differentiation.

56. The method of claim 55, wherein the fibroblast-like cells are produced for a therapeutic purpose.

57. A method of culturing cells, comprising culturing the cells in the presence of the fibroblast-like cells obtained by the method of claim 55; or conditioning cell culture media in the presence of the fibroblast-like cells and then culturing the cells therein; or culturing the cells in the conditioned cell culture media and in the presence of a population of the fibroblast-like cells.

58. The method of claim 57, wherein the cells being cultured comprise stem cells.

59. A self-feeder system for the growth of undifferentiated stem cells, said system comprising:
cultured cells of the stem cell line of claim 47;
wherein a population of the cells of the stem cell line differentiate into stem cell-derived fibroblast-like cells, whilst the remainder of the cells of the stem cell line remain in an undifferentiated pluripotent, multipotent or unipotent state, whereby the stem cell-derived fibroblast-like cells act as autogeneic feeder cells for the undifferentiated stem cells in culture.

60. A fibroblast-like cell line hESCdF-NCL as deposited at ECACC under Accession No. 04010601.

61. A method of culturing cells, comprising culturing the cells in the presence of cells of fibroblast-like cell line hESCdF-NCL of claim 60; or conditioning cell culture media in the presence of the hESCdF-NCL fibroblast-like cells, and then culturing the cells therein; or culturing the cells in the conditioned cell culture media and in the presence of a population of the hESCdF-NCL fibroblast-like cells.

62. The method of claim 61, wherein the cells being cultured comprise stem cells.


SEQUENCE LISTING (12 sequences)

SEQ ID NO: 1 - DNA, 18 bp, Artificial Sequence
  Chemically synthesized primer
gaaggtattcagccaaac

SEQ ID NO: 2 - DNA, 19 bp, Artificial Sequence
  Chemically synthesized primer
cttaatccaaaaaccctgg

SEQ ID NO: 3 - DNA, 23 bp, Artificial Sequence
  Chemically synthesized primer
gcgtacgcaaattaaagtccaga

SEQ ID NO: 4 - DNA, 25 bp, Artificial Sequence
  Chemically synthesized primer
cagcatcctaaacagctcgcagaat

SEQ ID NO: 5 - DNA, 36 bp, Artificial Sequence
  Chemically synthesized primer
gatcgggcccgccaccatgagtgtggatccagcttg

SEQ ID NO: 6 - DNA, 33 bp, Artificial Sequence
  Chemically synthesized primer
gatcgagctccatcttcacacgtcttcaggttg

SEQ ID NO: 7 - DNA, 20 bp, Artificial Sequence
  Chemically synthesized primer
ggagggagggggcaatgcac

SEQ ID NO: 8 - DNA, 17 bp, Artificial Sequence
  Chemically synthesized primer
ccccgagctcgcctact

SEQ ID NO: 9 - DNA, 22 bp, Artificial Sequence
  Chemically synthesized primer
cggaagagtgtctggagcaagt

SEQ ID NO: 10 - DNA, 21 bp, Artificial Sequence
  Chemically synthesized primer
gaacagtgccttcaccctcga

SEQ ID NO: 11 - DNA, 20 bp, Artificial Sequence
  Chemically synthesized primer
gtcagtggtggacctgacct

SEQ ID NO: 12 - DNA, 20 bp, Artificial Sequence
  Chemically synthesized primer
caccaccctgttgctgtagc